(19) United States
(12) Patent Application Publication
(10) Pub. No.: US 2009/0257548 A1
(43) Pub. Date: Oct. 15, 2009

(54) COMPUTED TOMOGRAPHY SYSTEM

(76) Inventors: Ashutosh Joshi, Waukesha, WI (US); Joseph James Lacey, Cambridge, WI (US)

Correspondence Address: PETER VOGEL;GE HEALTHCARE, 20225 WATER TOWER BLVD., MAIL STOP W492, BROOKFIELD, WI, 53045, US

(21) Appl. No.: 12/102,088

(22) Filed: Apr. 14, 2008

(51) Int. Cl. A61B 6/00; H01J 35/12

(52) U.S. Cl. 378/4; 378/200

(57) ABSTRACT

A computed tomography system is disclosed herein. The computed tomography system includes a detector module and a rail in contact with the detector module. The rail at least partially defines a passageway adapted to transfer a coolant

### BACKGROUND OF THE INVENTION

The subject matter disclosed herein relates to a computed tomography system. The computed tomography system is adapted to transfer a coolant in order to remove heat from a detector assembly.

Typically, in computed tomography (CT) systems, an x-ray source emits an x-ray beam toward a subject or object, such as a patient or a piece of luggage, positioned on a support. The x-ray beam, after being attenuated by the object, impinges upon the detector assembly. The intensity of the attenuated x-ray beam received at the detector assembly is typically dependent upon the attenuation of the x-ray beam by the object.

In known third generation CT systems, the x-ray source and the detector assembly are rotated on a rotatable gantry portion around the object to be imaged so that a gantry angle at which the x-ray beam intersects the object constantly changes. The detector assembly typically includes a plurality of detector modules. Each detector module typically comprises a substrate, a scintillator, a photodiode layer, and a plurality of electronic components. Additionally, the detector module is typically divided into a plurality of detector elements. Data representing the intensity of the received x-ray beam at each of the detector elements are collected across a range of gantry angles. The data are ultimately processed to form an image.

The electronic components produce heat that may cause a degradation in image quality through multiple mechanisms. For example, the gain of the photodiode layer is highly temperature dependent and operating the detector module at too high of a temperature may lead to image artifacts such as spots or rings. Also, the amount of pixel-to-pixel leakage between photodiodes increases with temperature. A high level of pixel-to-pixel leakage negatively impacts the signal-to-noise ratio within the detector module and results in reduced image quality. Also, an increase in the temperature of the detector module may result in problems with the mechanical alignment of the detector assembly and a collimator. Third generation CT imaging systems rely on an accurately aligned collimator to effectively block scattered x-rays. However, the mechanical alignment of the detector assembly and the collimator may change as the temperature increases outside of an optimal operating range. If the collimator is not properly aligned with the detector assembly, the result may be additional image artifacts.

The problem is that excessive heat within the detector assembly may lead to image artifacts from multiple sources, resulting in images of diminished quality.

### BRIEF DESCRIPTION OF THE INVENTION

The above-mentioned shortcomings, disadvantages and problems are addressed herein which will be understood by reading and understanding the following specification.

In an embodiment, a computed tomography system includes a detector module and a rail in contact with the detector module, the rail at least partially defining a passageway adapted to transfer a coolant.

In another embodiment, a computed tomography system includes a detector module and a rail attached to the detector module. The computed tomography system also includes a member attached to the rail, the member at least partially defining a passageway adapted to transfer a coolant.

In another embodiment, a computed tomography system includes a detector module including an electronic component. The computed tomography system includes a coolant in direct contact with the electronic component. The computed tomography system also includes a housing at least partially surrounding the electronic component, the housing adapted to transfer the coolant.

Various other features, objects, and advantages of the invention will be made apparent to those skilled in the art from the accompanying drawings and detailed description thereof.

### BRIEF DESCRIPTION OF THE DRAWINGS

FIG. 1 is a schematic diagram illustrating a CT system in accordance with an embodiment;

FIG. 2 is a schematic diagram illustrating a portion of a detector assembly attached to a pair of rails and a heat exchanger in accordance with an embodiment;

FIG. 3 is a schematic diagram illustrating a portion of a detector assembly attached to a pair of rails and a heat exchanger in accordance with another embodiment; and

FIG. 4 is a schematic diagram illustrating a cross section of a detector assembly attached to a pair of rails in accordance with another embodiment.

### DETAILED DESCRIPTION OF THE INVENTION

In the following detailed description, reference is made to the accompanying drawings that form a part hereof, and in which is shown by way of illustration specific embodiments that may be practiced. These embodiments are described in sufficient detail to enable those skilled in the art to practice the embodiments, and it is to be understood that other embodiments may be utilized and that logical, mechanical, electrical and other changes may be made without departing from the scope of the embodiments. The following detailed description is, therefore, not to be taken as limiting the scope of the invention.

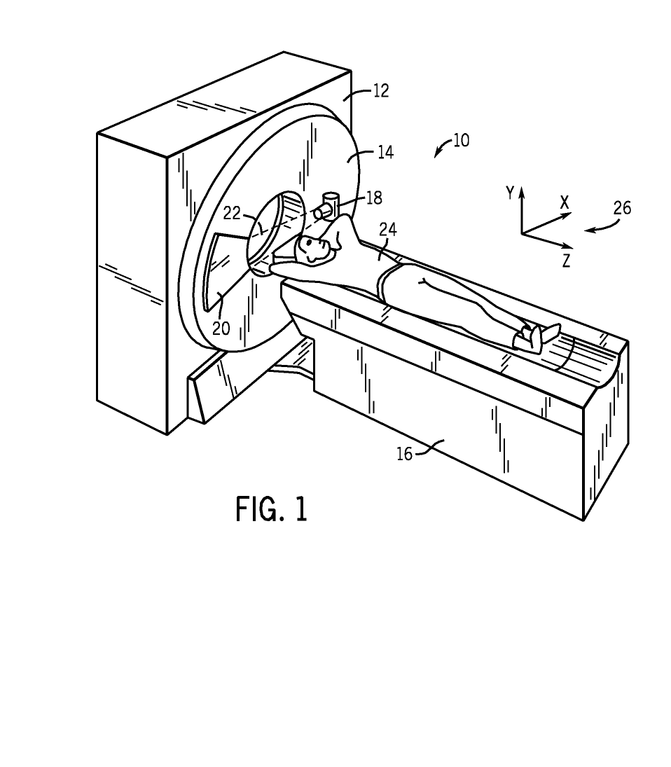

Referring to FIG. 1, a schematic representation of a computed tomography (CT) system 10 according to an embodiment is shown. The CT system 10 includes a gantry 12, a rotatable gantry portion 14, and a support 16. The rotatable gantry portion 14 is adapted to retain an x-ray source 18 and a detector assembly 20. The x-ray source 18 is configured to emit an x-ray beam 22 towards the detector assembly 20. The support 16 is configured to support a subject 24 being scanned. Hereinafter, the terms “subject” and “object” shall include anything capable of being imaged. The support 16 is capable of translating the subject 24 along a z-direction with respect to the gantry 12 as indicated by a coordinate axis 26.

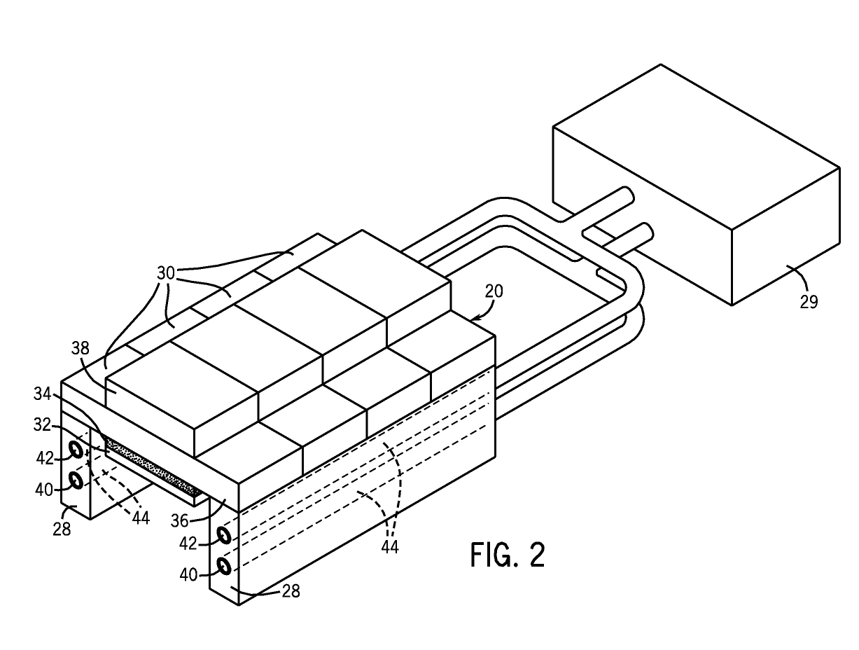

Referring to FIG. 2, a schematic representation of a portion of the detector assembly 20 attached to a pair of rails 28 and a heat exchanger 29 is shown in accordance with an embodiment. The detector assembly 20 is comprised of a plurality of detector modules 30. There are four detector modules 30 schematically represented in FIG. 2. Each detector module 30 includes a scintillator 32, a photodiode layer 34, a substrate 36, and one or more electronic components 38. The scintillator 32 converts received x-rays into visible light. The photodiode layer 34 is mounted radially outward of the scintillator 32 and converts the visible light from the scintillator 32 into an electrical signal. The substrate 36 provides a generally rigid mounting surface for the scintillator 32, the photodiode layer 34 and the electronic component 38. The scintillator 32 and the photodiode layer 34 are mounted to the radially inner side of the substrate 36. The electronic component 38 may comprise a component from the following nonlimiting list: an analog-to-digital converter (not shown) for converting the analog electrical signals from the photodiode into digital signals, a field-programmable gate-array (not shown), a power supply (not shown), and a voltage regulator (not shown). The analog-to-digital converter, the field-programmable gate-array, and the power supply are all well-known by those skilled in the art. The electronic component 38 is mounted radially outward from the substrate 36 for each detector module 30.

The substrate 36 of each detector module is attached to the rails 28. FIG. 2 schematically represents an embodiment where each rail 28 defines an inner passageway 40 and an outer passageway 42 through which a coolant 44 may flow. While this embodiment shows the inner passageway 40 and the outer passageway 42 defined by each of the rails 28, it should be appreciated that embodiments may include only one passageway 40, 42 defined by one of the rails 28 and embodiments may also include more than two passageways 40, 42 defined by each of the rails 28. The passageways 40, 42 are conductively coupled to the detector modules 30. For the purposes of this disclosure, the term “conductively coupled” is defined to include two components that are connected by a material that conducts heat. It should be understood that while the inner passageway 40 and the outer passageway 42 shown in FIG. 2 are round in cross-section and generally parallel to the rails 28, the passageways 40, 42 could be of any shape. A non-limiting list of passageway 40, 42 shapes includes: generally parallel to the rail 28; generally straight; serpentine; and shapes that vary in cross-section throughout the length of the rail 28. Additionally, it should be understood that the inner passageway 40 does not need to be of the same size and shape as the outer passageway 42.

The passageways 40, 42 defined by the rails 28 are in fluid communication with the heat exchanger 29. The coolant 44 is caused to circulate by a mechanical device such as a pump (not shown). For example, according to an embodiment, heat originating in the electronic components 38 conductively travels through the substrate 36 into the rail 28. After reaching the rail 28, heat from the electronic components 38 is absorbed by the coolant 44 circulating through the outer passageway 42. After absorbing heat, the coolant 44 flows from the outer passageway 42 to the inner passageway 40 through a connecting piece of hose (not shown). The coolant 44 then flows through the inner passageway 40 in generally the opposite direction as the coolant 44 had flowed in the outer passageway 42. While flowing through the inner passageway 40, the coolant 44 absorbs additional heat from the electronic components 38. The coolant 44 then flows to the heat exchanger 29 mounted to the rotatable gantry portion 14 (shown in FIG. 1). The heat exchanger 29 contains a structure with a large surface area to facility heat transfer as is well-known by those skilled in the art. The temperature of the coolant 44 is lowered while passing through the heat exchanger 29. After the coolant 44 has been cooled, it is pumped back through the outer passageway 42, where it can absorb more heat from the detector modules 30.

While the embodiment shown in FIG. 2 depicts the inner passageway 40 and the outer passageway 42 defined by the rail 28, embodiments may also be envisioned where the rail 28 only partially defines the passageways 40, 42. One example of an embodiment where the rail 28 only partially defines the passageways 40, 42 is where one side of the passageways 40, 42 is defined by a plate or cover (not shown) mounted to the rail 28. Additionally, it should be understood that embodiments may use a different layout in terms of how the coolant 44 is circulated through the rails 28. Considerations such as the expected temperature of the detector module 30, cost, and ease of manufacturing may be taken into account when determining the exact design of the one or more passageways 40, 42.

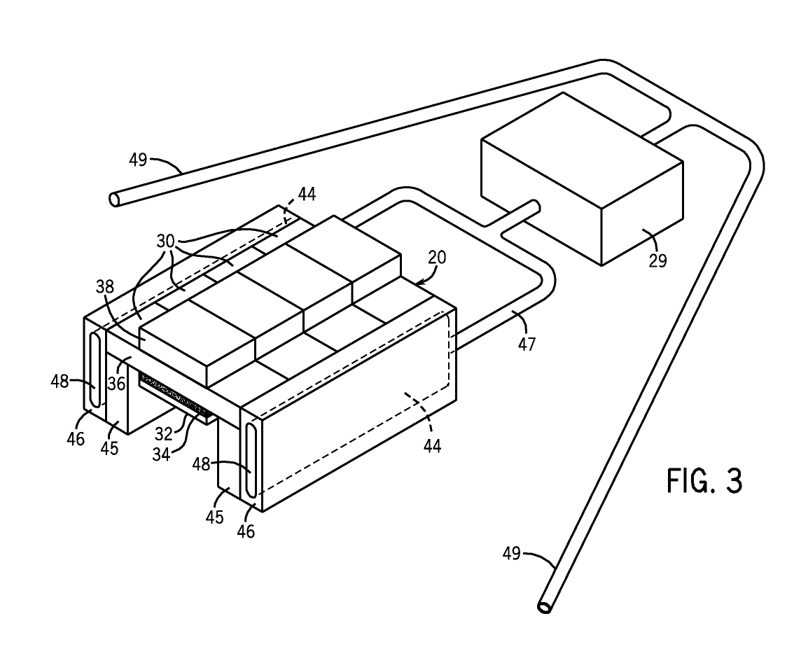

Referring to FIG. 3, a schematic representation of a portion of the detector assembly 20 attached to a pair of rails 45 and a heat exchanger 29 is shown in accordance with an embodiment. Common reference numbers are used to identify components that are generally identical to those of FIG. 2.

FIG. 3 shows a section of the detector assembly 20 with schematic representations of four detector modules 30. The pair of generally parallel rails 45 are attached to the substrate 36. Attached to the outer side of each rail 45 is a member 46. The member 46 is configured to define a passageway 48 that is adapted to transfer the coolant 44. Each member 46 may be permanently attached to the rail 45 by a process such as bonding or welding, or the member 46 may be removably attached by a bolt, fastener, or other type of removable mounting mechanism (not shown) to facilitate servicing of the detector assembly 20. The passageway 48 is conductively coupled to the detector modules 30. It should be understood that while the passageway 48 shown in the embodiment schematically illustrated in FIG. 3 is generally oval in cross-section and generally parallel to the rails 45, the passageway 48 could be of any shape. A non-limiting list of passageway 48 shapes includes: generally parallel to the rail 45; generally straight; serpentine; and shapes that vary in cross-section along the length of the member 46.

While the embodiment shown in FIG. 3 shows one passageway 48 defined by each of the members 46, it should be appreciated by those skilled in the art that embodiments could include either one passageway 48 defined by only one of the members 46 or embodiments could also include a plurality of passageways 48 defined by each of the members 46. The passageways 48 defined by the members 46 in FIG. 3 are in fluid communication with the heat exchanger 29. The coolant 44 is caused to circulate by a mechanical device such as a pump (not shown). For example, according to an embodiment, heat originating in the electronic components 38 conductively travels through the substrate 36. Once in the substrate 36, the heat travels either directly into the member 46, or else the heat travels through the rail 45 and then into the member 46. After reaching the member 46, heat from the electronic components 38 is absorbed by the coolant 44 circulating through the passageway 48, thus lowering the temperature of the electronic components 38. After absorbing heat, the coolant 44 flows through a hose 47 to the heat exchanger 29. The heat exchanger 29 contains a structure with a large surface area to facility heat transfer as is well-known by those skilled in the art. The temperature of the coolant 44 is lowered after passing through the heat exchanger 29. After the coolant 44 has been cooled, it is pumped back through a hose 49 and then back into the passageway 48 defined by the member 46, where it can absorb more heat from the electronic components 38. The connection between the hose 49 and the passageway 48 is not shown in FIG. 3. The heat exchanger is mounted to the rotatable gantry portion 14 (shown in FIG. 1). It should be understood that embodiments may circulate the coolant in a manner other than that shown in FIG. 3.

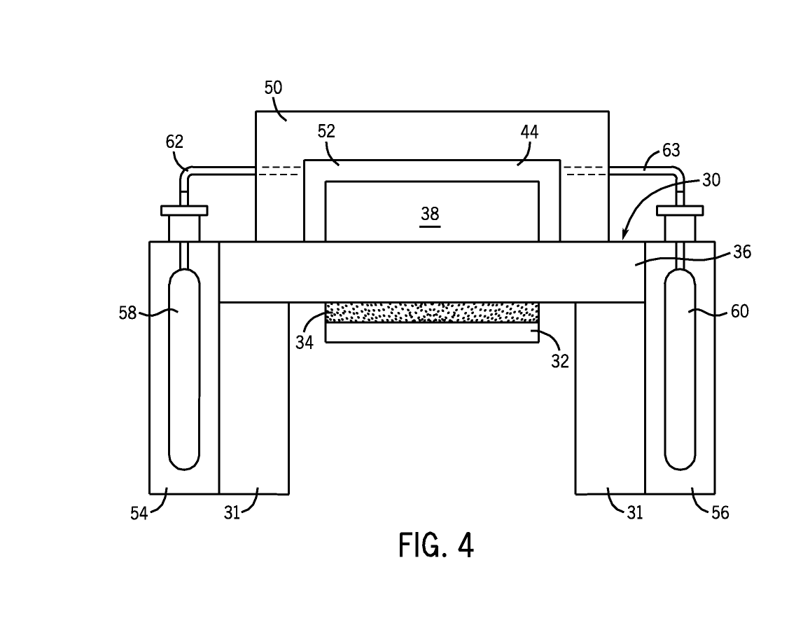

Referring to FIG. 4, a schematic representation of the cross section of the detector module 30 attached to a pair of rails 31 is shown in accordance with an embodiment. The embodiment shown in FIG. 4 is intended to be a non-limiting exemplary embodiment for illustrative purposes. Common reference numbers are used to identify components that are generally identical to those of FIG. 2 and FIG. 3.

The embodiment shown in FIG. 4 includes a housing 50 attached to the substrate 36 and partially surrounding the electronic component 38. The housing 50 is shaped in a manner so that the housing 50 and the substrate 36 define a passageway 52 adapted to transfer the coolant 44. Additionally, it should be understood that the electronic component 38 may not be mounted directly to the substrate 36. For example, according to an embodiment, the electronic component 38 may be mounted to the housing 50 instead of the substrate 36.

The embodiment shown in FIG. 4 also includes a first member 54 and a second member 56 attached to the pair of rails 31. The first member 54 defines an inflow passageway 58 and the second member 56 defines an outflow passageway 60. The inflow passageway 58 and the outflow passageway 60 are in fluid communication with the passageway 52 via a first hose 62 and a second hose 63. Coolant 44 is supplied to the inflow passageway 58. The coolant 44 flows from the inflow passageway 58 through the first hose 62 and into the passageway 52. Once in the passageway 52, the coolant 44 absorbs heat from the electronic component 38. After absorbing heat, the coolant 44 flows through the second hose 63 and into the outflow passageway 60 defined by the second member 56. In the embodiment illustrated in FIG. 4, the housing 50 defines a separate passageway 52 over each of the detector modules 30. However, it should be appreciated that the housing 50 may be shaped so that the electronic components 38 from multiple detector modules 30 fit inside a single passageway 52 according to an embodiment. Also, according to another embodiment, the coolant 44 may enter directly into the passageway 52 defined by the housing 50. Additionally, the coolant 44 may pass through a passageway (not shown) defined by the rail 31. After the coolant 44 has absorbed heat from the electronic component 38 and flowed to the outflow passageway 60 defined by the second member 56, the coolant 44 flows to a heat exchanger (not shown) where the coolant 44 is cooled before entering back into the inflow passageway 58 defined by member 54. The portion of the hydraulic circuit connecting the outflow passageway 60 to the heat exchanger and the heat exchanger to the inflow passageway 58 is not shown as it is well-known by those skilled in the art. Additionally, it should be understood that embodiments may circulate the coolant 44 through the passageway 52 defined by the housing 50 in a manner other than that shown in FIG. 4.

Referring now to FIGS. 2, 3, and 4, the coolant 44 may comprise any one of a number of well-known coolants. A non-limiting list of a well-known coolants includes water glycol, mineral oil, and dielectric fluids such as dielectric oil and perfluorocarbon fluid. Other coolants may be employed as well. The particular coolant 44 chosen may depend on the specifics of the application. For example, the range of operating temperatures, the materials used for the rails 28, 45, 31, the substrate 36, or the member 46, 54, 56 may also affect the choice of coolant 44. Additionally, for embodiments where the coolant 44 is in direct contact with the electronic component 38 such as that shown in FIG. 4, it may be desirable to choose a coolant 44 with dielectric properties to prevent a short-circuit. Additionally, using a coolant 44 and a heat exchanger 29 may manage the temperature of the detector modules 30 and the rails 28, 45, 31 more effectively, enabling the use of a less expensive material to build the rails 28, 45, 31. For example, if the operating temperature of the CT system 10 (shown in FIG. 1) is more closely controlled, it may be possible to use a rail material with a higher coefficient of thermal expansion. For example, conventional CT systems typically use rails made from steel. Embodiments may be able to use a material such as an aluminum alloy, or an aluminum silicon carbide. Some of these rail materials may provide an additional advantage by having a higher stiffness-to-weight ratio than steel, thus enabling a lighter rotatable gantry portion 14 (shown in FIG. 1).

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1. A computed tomography system comprising:
a rotatable gantry portion;
a detector module; and
a pair of generally parallel rails connecting the detector module to the rotatable gantry portion, said pair of generally parallel rails engaging the detector module on generally opposite sides of the detector module and one of said pair of generally parallel rails at least partially defining a passageway adapted to transfer a coolant.

2. The computed tomography system of claim 1, wherein the coolant is selected from the group consisting of water glycol, mineral oil, dielectric oil and perfluorocarbon fluid.

3. The computed tomography system of claim 1, further comprising a heat exchanger in fluid communication with the passageway.

4. The computed tomography system of claim 1, wherein said one of said pair of generally parallel rails completely defines the passageway adapted to transfer the coolant.

5. The computed tomography system of claim 1, wherein said one of said pair of generally parallel rails comprises an aluminum alloy.

6. The computed tomography system of claim 1, wherein said one of said pair of generally parallel rails comprises an aluminum silicon carbide.

7. A computed tomography system comprising:
a detector module comprising a first surface and a second surface, the first surface and the second surface being disposed at generally opposite end portions of the detector module;
a first rail attached to the first surface of the detector module;
a second rail attached to the second surface of the detector module;
a first member attached to the first rail, the first member at least partially defining a first passageway adapted to transfer a coolant; and
a second member attached to the second rail, the second member at least partially defining a second passageway adapted to transfer the coolant.

8. The computed tomography system of claim 7, wherein the coolant is selected from the group consisting of water glycol, mineral oil, dielectric oil and perfluorocarbon fluid.

9. The computed tomography system of claim 7, wherein the first rail and the first member collectively defines the first passageway.

10. The computed tomography system of claim 7, wherein the first member is a plate.

11. The computed tomography system of claim 8, wherein the first member is attached to the first rail and to a substrate of the detector module.

12. The computed tomography system of claim 7, wherein the first member comprises either an aluminum alloy or an aluminum silicon carbide.

13. The computed tomography system of claim 7, further comprising a heat exchanger in fluid communication with the first passageway and the second passageway.

14. The computed tomography system of claim 7, wherein the first member is removably attached to the first rail.

15. The computed tomography system of claim 7, wherein the first member is brazed to the first rail.

16. A computed tomography system comprising:
a detector module including an electronic component;
a coolant in direct contact with the electronic component; and
a housing at least partially surrounding the electronic component, the housing adapted to transfer the coolant.

17. The computed tomography system of claim 16, further comprising a heat exchanger connected to the housing.

18. The computed tomography system of claim 16, wherein the coolant comprises a dielectric fluid.

19. The computed tomography system of claim 18, wherein the dielectric fluid comprises a dielectric oil or a perfluorocarbon fluid.
